(19) United States
(12) Patent Application Publication
(10) Pub. No.: US 2007/0088069 A1
(43) Pub. Date: Apr. 19, 2007

(54) Novel imidazoles

(75) Inventors: Xue-Min Cheng, Ann Arbor, MI (US); Richard H. Hutchings, Ann Arbor, MI (US); Walter Allen Howard, Parma, MI (US); Robert Michael Kennedy, Ann Arbor, MI (US); William Keun-Chan Park, Ann Arbor, MI (US); Yuntao Song, Ann Arbor, MI (US); Bharat K. Trivedi, Farmington Hills, MI (US)

Correspondence Address: WARNER-LAMBERT COMPANY, 2800 PLYMOUTH RD, ANN ARBOR, MI, 48105, US

(73) Assignee: Warner-Lambert Company LLC

(21) Appl. No.: 11/546,168

(22) Filed: Oct. 11, 2006

Related U.S. Application Data
(60) Provisional application No. 60/726,920, filed on Oct. 14, 2005.

(51) Int. Cl. A61K 31/4164; C07D 233/66

(52) U.S. Cl. 514/396; 548/331.5

(57) ABSTRACT

Novel imidazole compounds and pharmaceutical compositions are described, as are methods of using such compounds, alone or in combination with another pharmaceutically active agent, to treat subjects, including humans, suffering from hyperlipidemia, hypercholesterolemia, hypertriglyceridemia, and atherosclerosis.

### CROSS REFERENCE

This application claims benefit of U.S. Provisional Application Ser. No. 60/726,920 filed Oct. 14, 2005.

### BACKGROUND OF THE INVENTION

High levels of blood cholesterol and blood lipids are conditions involved in the onset of atherosclerosis. The conversion of HMG-CoA to mevalonate is an early and rate-limiting step in the cholesterol biosynthetic pathway. This step is catalyzed by the enzyme HMG-CoA reductase. It is known that inhibitors of HMG-CoA reductase are effective in lowering the blood plasma level of low density lipoprotein cholesterol (LDL-C), in man. (cf. M. S. Brown and J. L. Goldstein, New England Journal of Medicine, 305, No. 9, 515-517 (1981)). It has been established that lowering LDL-C levels affords protection from coronary heart disease (cf. Journal of the American Medical Association, 251, No. 3, 351-374 (1984)).

Statins are collectively lipid lowering agents. Representative statins include atorvastatin, lovastatin, pravastatin, simvastatin and rosuvastatin. Atorvastatin and pharmaceutically acceptable salts thereof are selective, competitive inhibitors of HMG-CoA reductase. A number of patents have issued disclosing atorvastatin. These include: U.S. Pat. Nos. 4,681,893; 5,273,995 and 5,969,156, which are incorporated herein by reference.

All statins interfere, to varying degrees, with the conversion of HMG-CoA to the cholesterol precursor mevalonate by HMG-CoA reductase. These drugs share many features, but also exhibit differences in pharmacologic attributes that may contribute to differences in clinical utility and effectiveness in modifying lipid risk factors for coronary heart disease. (Clin. Cardiol. Bol. 26 (Suppl. III), III-32-III-38 (2003)). Some of the desirable pharmacologic features with statin therapy include potent reversible inhibition of HMG-CoA reductase, the ability to produce large reductions in LDL-C and non-high-density lipoprotein cholesterol (non-HDL-C), the ability to increase HDL cholesterol (HDL-C), tissue selectivity, optimal pharmacokinetics, availability of once a day dosing and a low potential for drug-drug interactions. Also desirable is the ability to lower circulating very-low-density-lipoprotein (VLDL) as well as the ability to lower triglyceride levels.

At the present time, the most potent statins display in vitro IC₅₀ values, using purified human HMG-CoA reductase catalytic domain preparations, of between about 5.4 and about 8.0 nM. (Am. J. Cardiol. 2001; 87(suppl): 28B-32B; Atheroscer Suppl. 2002; 2:33-37). Generally, the most potent LDL-C-lowering statins are also the most potent non-HDL-C-lowering statins. Thus, maximum inhibitory activity is desirable. With respect to HDL-C, the known statins generally produce only modest increases in HDL-C. Therefore, the ability to effect greater increases in HDL-C would be advantageous as well.

With respect to tissue selectivity, differences among statins in relative lipophilicity or hydrophilicity may influence drug kinetics and tissue selectivity. Relatively hydrophilic drugs may exhibit reduced access to nonhepatic cells as a result of low passive diffusion and increased relative hepatic cell uptake through selective organic ion transport. In addition, the relative water solubility of a drug may reduce the need for extensive cytochrome P450 (CYP) enzyme metabolism. Many drugs, including the known statins, are metabolized by the CYP3A4 enzyme system. (Arch. Intern. Med. 2000; 160:2273-2280; J. Am. Pharm. Assoc. 2000; 40:637-644). Thus, relative hydrophilicity is desirable with statin therapy.

Two important pharmacokinetic variables for statins are bioavailability and elimination half-life. It would be advantageous to have a statin with limited systemic availability so as to minimize any potential risk of systemic adverse effects, while at the same time having enough systemic availability so that any pleiotropic effects can be observed in the vasculature with statin treatment. These pleiotropic effects include improving or restoring endothelial function, enhancing the stability of atherosclerotic plaques, reduction in blood plasma levels of certain markers of inflammation such as C-reactive protein, decreasing oxidative stress and reducing vascular inflammation. (Arterioscier. Thromb. Vasc. Biol. 2001; 21:1712-1719; Heart Dis. 5(1):2-7, 2003). Further, it would be advantageous to have a statin with a long enough elimination half-life to maximize effectiveness for lowering LDL-C.

Finally, it would be advantageous to have a statin that is either not metabolized or minimally metabolized by the CYP 3A4 systems so as to minimize any potential risk of drug-drug interactions when statins are given in combination with other drugs.

Accordingly, it would be most beneficial to provide a statin having a combination of desirable properties including high potency in inhibiting HMG-CoA reductase, the ability to produce large reductions in LDL-C and non-high density lipoprotein cholesterol, the ability to increase HDL cholesterol, selectivity of effect or uptake in hepatic cells, optimal systemic bioavailability, prolonged elimination half-life, and absence or minimal metabolism via the CYP3A4 system.

### SUMMARY OF THE INVENTION

This invention provides a novel series of imidazoles. Compounds of the invention are potent inhibitors of cholesterol biosynthesis. Accordingly, the compounds find utility as therapeutic agents to treat hyperlipidemia, hypercholesterolemia, hypertriglyceridemia and atherosclerosis. More specifically, the present invention provides a compound having a Formula I, 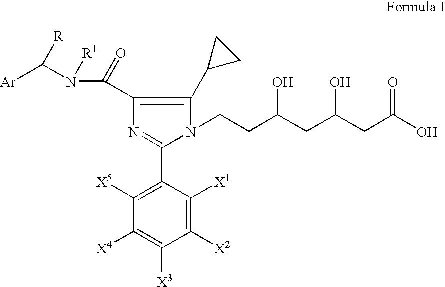
or a pharmaceutically acceptable salt, ester, amide, or stereoisomer thereof, wherein
Ar is an unsubstituted or substituted phenyl;
R¹ is H or C₁-C₄ alkyl;
R¹ is H or methyl;
and X¹, X², X³, X⁴, and X⁵ are each independently selected from the group consisting of H, F, and Cl.

The present invention further provides a compound having a Formula II, 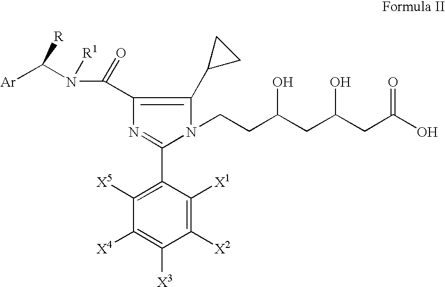
or a pharmaceutically acceptable salt, ester, amide, or stereoisomer thereof, wherein
Ar is an unsubstituted or substituted phenyl;
R is C₁-C₄ alkyl;
R¹ is H or methyl; and
X¹, X², X³, X⁴, and X⁵ are each independently selected from the group consisting of H, F and Cl.

Further provided is a compound having a Formula III, 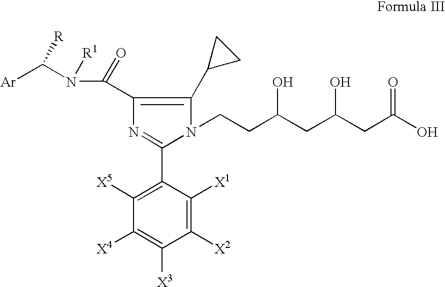
or a pharmaceutically acceptable salt, ester, amide, or stereoisomer thereof, wherein
Ar is an unsubstituted or substituted phenyl;
R is C₁-C₄ alkyl;
R¹ is H or methyl; and
X¹, X², X³, X⁴, and X⁵ are each independently selected from the group consisting of H, F and Cl.

### DETAILED DESCRIPTION

The present invention provides a compound having a Formula I, 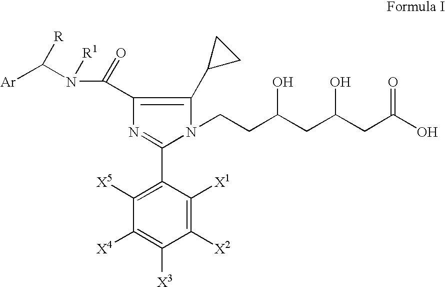
or a pharmaceutically acceptable salt, ester, amide, or stereoisomer thereof, wherein
Ar, R, R¹, X¹, X², X³, X⁴, and X⁵ are as defined above.

Further provided is the above compound wherein X¹, X², X⁴, and X⁵ are H; and X³ is F.

Further provided is the compound wherein R is methyl.

The present invention provides inter alia the following compounds:
(3R,5R)-7-[5-Cyclopropyl-2-(4-fluoro-phenyl)-4-((R)-1-phenyl-ethylcarbamoyl)-imidazol-1-yl]-3,5-dihydroxy-heptanoic acid;
(3R,5R)-7-[5-Cyclopropyl-2-(4-fluoro-phenyl)-4-((S)-1-phenyl-ethylcarbamoyl)-imidazol-1-yl]-3,5-dihydroxy-heptanoic acid;
(3R,5R)-7-(5-Cyclopropyl-2-(4-fluoro-phenyl)-4-[methyl-((R)-1-phenyl-ethyl)-carbamoyl]-imidazol-1-yl)-3,5-dihydroxy-heptanoic acid;
7-[5-Cyclpropyl-2-(4-fluoro-phenyl)-4-(4-methyl-benzylcarbamoyl)-imidazol-1-yl]-3,5-dihydroxy-heptanoic acid;
7-[5-Cyclopropyl-2-(4-fluoro-phenyl)-4-(4-methoxy-benzylcabamoyl)-imidazol-1-yl]-3,5-dihydroxy-heptanoic acid;
   and pharmaceutically acceptable salts and lactone forms thereof.

Further provided is the above-described compound wherein Ar is substituted by one of more groups selected from: (C₁-C₆)alkoxy, (C₁-C₆)alkyl, F and Cl.

Further provided is a stereoisomer of the above-described compound comprising a (3R,5R)-isomer or the pharmaceutically acceptable salt, ester, or amide thereof.

Further provided is a stereoisomer of the above compound comprising a (3S,5R)-isomer or the pharmaceutically acceptable salt, ester, or amide thereof.

Further provided is a stereoisomer of the above compound comprising a (3R,5S)-isomer or the pharmaceutically acceptable salt, ester, or amide thereof.

Further provided is a stereoisomer of the above compound comprising a (3S,5S)-isomer or the pharmaceutically acceptable salt, ester or amide thereof.

The present invention further provides a compound having a Formula II, 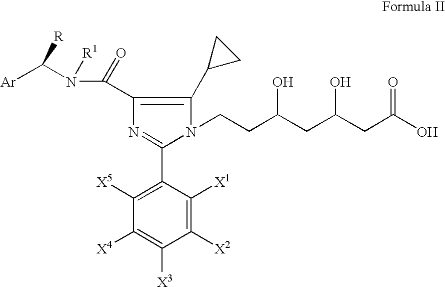
or a pharmaceutically acceptable salt, ester, amide, or stereoisomer thereof, wherein
Ar, R, R¹, X¹, X², X³, X⁴, and are as defined above.

Further provided is a compound having a Formula III, 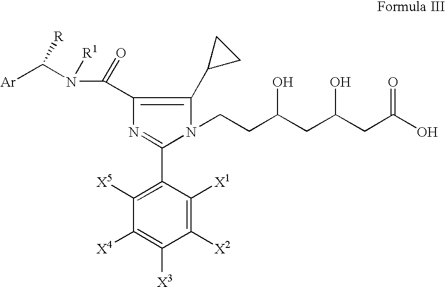
or a pharmaceutically acceptable salt, ester, amide, or stereoisomer thereof, wherein
Ar, R, R¹, X¹, X², X³, X⁴, and X⁵ are as defined above.

Further provided is a pharmaceutical composition comprising a the above compounds.

Further provided is a method of inhibiting cholesterol biosynthesis in a mammal.

Further provided is a method of lowering LDL cholesterol in a mammal.

Further provided is a method of raising HDL cholesterol in a mammal.

Further provided is a method of treating, preventing or controlling hyperlipidemia in a mammal.

Further provided is a method of treating, preventing or controlling hypercholesterolemia in a mammal.

Further provided is a method of treating, preventing or controlling hypertriglyceridemia in a mammal.

Further provided is a method of treating, preventing or controlling atherosclerosis in a mammal.

The present invention further encompasses each of the title compounds set forth in the Examples herein.

The term “alkyl” as used herein refers to a straight or branched hydrocarbon of from 1 to 11 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, —O(CH₂)₀₋₂CF₃, -Oaryl, halogen, nitro, cyano, ═O, ═S, —OH, —SH, —CF₃, —CO₂H, —CO₂C₁-C₆ alkyl, —NR′R″, NR′SO₂R″, NR′CONR′R″, or —CONR′R″ where R′ and R″ are independently H, alkyl, cycloalkyl, alkenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl).

The term “lower alkyl” as used herein refers to a subset of alkyl which means a straight or branched hydrocarbon radical having from 1 to 6 carbon atoms and includes, for example, methyl, ethyl, n-propyl, isopropyl, n-butyl, sec-butyl, isobutyl, tert-butyl, n-pentyl, n-hexyl, and the like. Optionally, lower alkyl is referred to as “C₁-C₆alkyl.”

The term “haloalkyl” as used herein refers to a lower alkyl radical, as defined above, bearing at least one halogen substituent, for example, chloromethyl, fluoroethyl, trifluoromethyl, or 1,1,1-trifluoroethyl and the like. Haloalkyl can also include perfluoroalkyl wherein all hydrogens of a lower alkyl group are replaced with fluorine atoms.

The term “alkenyl” means a straight or branched unsaturated hydrocarbon radical from 2 to 12 carbon atoms and includes, for example, ethenyl, 1-propenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, 1-dodecenyl, and the like.

The term “alkynyl” means a straight or branched hydrocarbon radical of 2 to 12 carbon atoms having at least one triple bond and includes, for example, 3-propynyl, 1-butynyl, 3-butynyl, 1-pentynyl, 3-pentynyl, 3-methyl-3-butynyl, 1-hexynyl, 3-hexynyl, 3-hexynyl, 3-heptynyl, 1-octynyl, 1-nonynyl, 1-decynyl, 1-undecynyl, 1-dodecynyl, and the like.

The term “alkylene” as used herein refers to a divalent group derived from a straight or branched chain saturated hydrocarbon having from 1 to 10 carbon atoms by the removal of two hydrogen atoms, for example methylene, 1,2-ethylene, 1,1-ethylene, 1,3-propylene, 2,2-dimethylpropylene, and the like. The alkylene groups of this invention can be optionally substituted with one or more of the substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, NR′R″, or —CONR′R″, where R′ and R″ are independently H, alkyl, cycloalkyl, alkenyl, alkynyl, aryl, aralkyl, heteroaryl, heteroaralkyl, or joined together to form a 4 to 7 member ring; or N, R′ and R″ taken together form a 4-7 member ring. Useful alkylene groups have from 1 to 6 carbon atoms (C₁-C₆ alkylene).

The term “heteroatom” as used herein represents oxygen, nitrogen, or sulfur (O, N, or S) as well as sulfoxyl or sulfonyl (SO or SO₂) unless otherwise indicated.

The term “hydrocarbon chain” as used herein refers to a straight hydrocarbon of from 2 to 6 carbon atoms. The hydrocarbon chain is optionally substituted with one or more substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, NR′R″ or —CONR′R″, where R′ and R″ are independently H, alkyl, cycloalkyl, alkenyl, alkynyl, aryl, aralkyl, heteroaryl, heteroaralkyl or joined together to form a 4 to 7 member ring; or N, R′ and R″ taken together form a 4-7 member ring.

The term “hydrocarbon-heteroatom chain” as used herein refers to a hydrocarbon chain wherein one or more carbon atoms are replaced with a heteroatom. The hydrocarbon-heteroatom chain is optionally substituted with one or more substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, NR′R″ or —CONR′R″, where R′ and R″ are independently H, alkyl, cycloalkyl, alkenyl, alkynyl, aryl, aralkyl, heteroaryl, heteroaralkyl or joined together to form a 4 to 7 member ring; or N, R′ and R″ taken together form a 4-7 member ring.

The term “heteroalkylene” as used herein, refers to an alkylene radical as defined above that includes one or more heteroatoms such as oxygen, sulfur, or nitrogen (with valence completed by hydrogen or oxygen) in the carbon chain or terminating the carbon chain.

The terms “lower alkoxy” and “lower thioalkoxy” as used herein refers to O-alkyl or S-alkyl of from 1 to 6 carbon atoms as defined above for “lower alkyl.”

The term “aryl” or “Ar” as used herein refers to an aromatic ring which is unsubstituted or optionally substituted by 1 to 4 substituents selected from lower alkyl, lower alkoxy, lower thioalkoxy, —O(CH₂)₀₋₂CF₃, -Oaryl, —OSO₂R′, nitro, cyano —OH, —SH, —CF₃, —CO₂H, —CO₂C₁-C₆ alkyl, —NR′R″, NR′SO₂R″, NR′CONR′R″, —SO₁₋₂alkyl, SO₁₋₂aryl, SO₂NR′R″, or —CONR′R″, where R′ and R″ are independently H, alkyl, cycloalkyl, alkenyl, alkynyl, aryl, aralkyl, heteroaryl, heteroaralkyl or joined together to form a 4 to 7 member ring; or N, R′ and R″ taken together form a 4-7 member ring. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, or the like. Further, the term “aryl” means a cyclic or polycyclic aromatic ring having from 5 to 12 carbon atoms, and being unsubstituted or substituted with up to 4 of the substituent groups recited above for alkyl, alkenyl, and alkynyl.

The term aralkyl as used herein means aryl, as defined above, attached to an alkyl group, as defined above.

The term “heteroaryl” means an aromatic ring containing one or more heteroatom. The heteroaryl is optionally substituted with one or more groups enumerated for aryl. Examples of heteroaryl include, but are not limited to thienyl, furanyl, pyrrolyl, pyridyl, pyrimidyl, imidazoyl, pyrazinyl, oxazolyl, thiazolyl, benzothienyl, benzofuranyl, indolyl, quinolinyl, isoquinolinyl, and quinazolinyl, and the like. Further, the term “heteroaryl” means an aromatic mono-, bi-, or polycyclic ring incorporating one or more (i.e. 1-4) heteroatoms selected from N, O, and S, which mono-, bi-, or polycyclic ring is optionally substituted with lower alkyl, lower alkoxy, lower thioalkoxy, —O(CH₂)₀₋₂CF₃, halogen, nitro, cyano —OH, —SH, —CF₃, —CO₂H, —CO₂C₁-C₆ alkyl, —NR′R″, —SO₂alkyl, SO₂aryl, SO₂NR′R″, or —CONR′R″, where R′ and R″ are independently H, alkyl, cycloalkyl, alkenyl, alkynyl, aryl, aralkyl, heteroaryl, heteroaralkyl or joined together to form a 4 to 7 member ring; or N, R′ and R″ taken together form a 4-7 member ring. Examples further include I-, 2-, 4-, or 5-imidazolyl, I-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 1, 3-, or 5-triazolyl, I-, 2-, or 3-tetrazolyl, 2-pyrazinyl, 2-, 4-, or 5-pyrimidinyl, I- or 2-piperazinyl, 2-, 3-, or 4-morpholinyl. Examples of suitable bicyclic heteroaryl compounds include, but are not limited to indolizinyl, isoindolyl, benzofuranyl, benzothienyl, benzoxazolyl, benzimidazolyl, quinolinyl, isoquinolinyl, quinazolinyl, I-, 2-, 3-, 4-, 5-, 6-, or 7-indolyl, I-, 2-, 3-, 5-, 6-, 7-, or 8-indolizinyl, I-, 2-, 3-, 4-, 5-, 6-, or 7-isoindolyl, 2-, 3-, 4-, 5-, 6-, or 7-benzothienyl, 2-, 4-, 5-, 6-, or 7-benzoxazolyl, I-, 2-, 4-, 5-, 6-, or 7-benzimidazolyl, 2-, 3-, 4-, 5-, 6-, 7-, or 8-quinolinyl, and I-, 3-, 4-, 5-, 6-, 7-, or 8-isoquinolinyl.

The term heteroaralkyl, as used herein, means heteroaryl, as defined above, attached to an alkyl group as defined above.

The term “heterocycle” means a saturated mono- or polycyclic (i.e. bicyclic) ring incorporating one or more (i.e. 1-4) heteroatoms selected from N, O, and S. It is understood that a heterocycle is optionally substituted with one or more of the substituents selected from lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NR′R″ or —CONR′R″ where R′ and R″ are independently H, alkyl, cycloalkyl, alkenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl). Examples of suitable monocyclic heterocycles include, but are not limited to piperidinyl, pyrrolidinyl, piperazinyl, azetidinyl, aziridinyl, morpholinyl, thietanyl, oxetaryl.

The term “ring” as used herein includes heteroaryl, cycloalkyl or aryl and further includes fused, monocyclic and polycyclic permutations thereof.

The term “cycloalkyl” means a saturated hydrocarbon ring. Further, the term “cycloalkyl” means a hydrocarbon ring containing from 3 to 12 carbon atoms, for example, cyclopropyl, cyclobutyl, cyclopentyl, cyclohexyl, cycloheptyl, cycloctyl, decalinyl, norpinanyl, or adamantyl. The cycloalkyl ring may be unsubstituted or substituted by 1 to 3 substituents selected from one or more of the substituents selected from lower alkoxy, lower thioalkoxy, —(CH₂)₀₋₂CF₃, halogen, nitro, cyano, ═O, ═S, —OH, —SH, —CF₃, —CO₂H, —CO₂C₁-C₆ alkyl, —NR′R″ or —CONR′R″ where R′ and R″ are independently H, alkyl, cycloalkyl, alkenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl), wherein alkyl, aryl, and heteroaryl are as defined herein. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “cycloalkenyl” means a cycloalkyl group having one or more carbon-carbon double bond. Example includes cyclobutene, cyclopentene, cyclohexene, cycloheptene, cyclobutadiene, cyclopentadiene, and the like.

The symbol “═” means a double bond.

When a bond is represented by a line such as “---” this is meant to represent that the bond may be absent or present provided that the resultant compound is stable and of satisfactory valency. If an asymmetric carbon is created by such a bond, a particular stereochemistry is not to be implied.

As used herein, the following terms have the meanings given: RT or rt means room temperature. MP means melting point. MS means mass spectroscopy. TLC means thin layer chromatography. [S]at. means saturated. [C]one. means concentrated. TBIA means [(4R,6R)-6-(2-Amino-ethyl)-2,2-dimethyl-[1,3]dioxan-4-yl]-acetic acid tert-butyl ester. DCM means dichloromethane, which is used interchangeably with methylene chloride. NBS means N-Bromosuccinimide. “h” means hour. “v/v,” means volume ratio or “volume per volume”. “R_{f}” means retention factor. “Tf₂O” or “TfO” means triflic anhydride or C(F)₃S(O)₂OS(O)₂C(F)₃. Ac₂O means acetic anhydride. “[T]rifluorotol.” Or “TFT” means trifluoro methyl-benzene. “DMF” means dimethylformamide. “DCE” means dichloroethane. “Bu” means butyl. “Me” means methyl. “Et” means ethyl. “DBU” means 1,8-Diazabicyclo-[5.4.0]undec-7-ene. “TBS” means “TBDMS” or tert-Butyldimethylsilyl. “DMSO” means dimethyl sulfoxide. “TBAF” means tetrabutylammonium fluoride. THF means tetrahydrofuran. n-BuLi or Buli means n-butyl lithium. TFA means trifluoroacetic acid. i-Pr means isopropyl. [M]in means minutes. ml or mL means milliliter. “M” or “m” means molar. “Bn” means benzyl. “PyBOP” means bromo-tris-pyrrolidino-phosphonium hexafluorophosphate. “OtBu” means t-butoxy. “Ts” or “Tosyl” means p-toluenesulfonyl. “PS-DIEA” means polystyrene-bound diisopropylethylamine. “PS-NCO” means polystyrene-bound isocyanate resin. “Ph” means phenyl. As used herein, “hydrogenolysis” means the cleaving of a chemical bond by hydrogen. “EDCl” or “EDC” means 1-(3-dimethylaminopropyl)-3-ethylcarbondiimide hydrochloride. “NMP” means 1-methyl-2-pyrrolidinone. “DPP” or “DPPA” means diphenyl phosphoryl azide. “HOBt” 1-hydroxybenzotriazole.

In some situations, compounds may exist as tautomers. All tautomers included within Formulas I-III are provided by this invention.

Certain compounds of the present invention can exist in unsolvated form as well as solvated form including hydrated form. In general, the solvated form including hydrated form is equivalent to the unsolvated form and is intended to be encompassed within the scope of the present invention.

The term “stereoisomer” as used herein refers to both geometric (e.g., cis and trans isomers) and/or optical isomers (e.g., R and S enantiomers) of a compound of the invention. Racemic, enantiomeric, diastereomeric, and epimeric mixtures of isomers are contemplated by the present invention.

Certain of the compounds of the present invention possess one or more chiral centers and each center may exist in the R or S configuration. Additionally, the compounds of the present invention may exist as geometric isomers. The present invention includes all cis, trans, syn, anti, entgegen (E), and zusammen (Z) isomers as well as the appropriate mixtures thereof.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula 1, 2, or 3 contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

The term “racemate” as used herein, is meant to include both the racemic compound wherein one homogeneous form is produced containing both enantiomers in equimolar amounts and the racemic mixture or conglomerate wherein two forms are produced in equimolar amounts each containing the single enantiomer. Such mixtures may be separated by conventional techniques known to those skilled in the art—see, for example, Stereochemistry of Organic Compounds by E. L. Eliel and S. H. Wilen (Wiley, New York, 1994).

When a bond to a substituent is shown to cross the bond(s) connecting 2 atoms in a ring, then such substituent may be bonded to any atom in the ring, provided the atom will accept the substituent without violating its valency. When there appears to be several atoms of the substituent that may bond to the ring atom, then it is the first atom of the listed substituent that is attached to the ring, unless indicated otherwise.

Unless indicated otherwise, “compound of the invention” or “compounds of the invention” includes the compound itself as well as pharmaceutically acceptable salts, esters, amides, hydrates, or stereoisomers thereof.

The term “patient” or “subject” means all animals and mammals, including humans. Examples of patients or subjects include humans, cows, dogs, cats, goats, sheep, pigs, and rabbits.

The phrases “effective amount” and “therapeutically effective amount” mean that amount of a compound of Formula 1, 2, or 3, and other pharmacological or therapeutic agents described below, that will elicit a biological or medical response in a tissue, system, animal, or mammal that is being sought by the administrator (such as a researcher, doctor, or veterinarian) which includes alleviation of the symptoms of the condition or disease being treated and the prevention, slowing or halting of progression of one or more conditions, for example vascular conditions such as hyperlipidemia, atherosclerosis, hypercholesterolemia, hypertriglyceridemia, sitosterolemia, vascular inflammation, and the like. As would be understood by a skilled artisan, a “therapeutically effective amount” will vary from subject to subject and will be determined on a case by case basis. Factors to consider include, but are not limited to, the subject being treated, weight, health, and compound administered.

The term “a pharmaceutically acceptable salt, ester, amide, hydrate, or stereoisomer” as used herein refers to those acid addition salts, base addition salts, esters, amides, hydrates, and stereoisomers (optical, geometric, and tautomeric) of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

Further, the term “a pharmaceutically acceptable salt” refers to the relatively non-toxic, inorganic and organic acid addition or base salts of compounds of the invention. These salts can be prepared in situ during the final isolation and purification of the compounds or by separately reacting the purified compound in its free form with a suitable organic or inorganic acid or base and isolating the salt thus formed. Representative anionic or acid addition salts include acetate, aspartate, besylate, bicarbonate, carbonate, camysylate, citrate, edisylate, fumarate, gluconate, hydrobromide, bromide, hydrochloride, chloride, D-lactate, L-lactate, malate, mesylate, pamoate, phosphate, succinate, sulphate, D-tartrate, L-tartrate, benzoate, gluceptate, glucuronate, hibenzate, isethionate, malonate, methylsulphate, 2-napsylate, nicotinate, nitrate, orotate, stearate, tosylate, adipate, arabogalactanesulphate, ascorbate, estolate, galacturonate, glutamate, hippurate, 3-hydroxy-2-naphthoate, 1-hydroxy-2-naphthoate, iodide, lactobionate, maleate, mandelate, mucate, napadisylate, oleate, oxalate, saccharate, salicylate, sulphosalicylate, cholate, and tryptophanate. (See, for example, Berge S. M., et al., “Pharmaceutical Salts,” J. Pharm. Sci., 1977; 66:1-19, which is incorporated herein by reference.) The free base form may be regenerated by contacting the salt form with a base. While the free base may differ from the salt form in terms of physical properties, such as solubility, the salts are equivalent to their respective free bases for the purposes of the present invention.

Representative cationic or base salts include calcium, choline, magnesium, potassium, sodium, aluminum, ammonium, quaternary ammonium, and amine cations including tetramethylammonium, tetraethylammonium, methylamine, dimethylamine, trimethylamine, triethylamine, ethylamine, and the like, arginine, benzathine, diethylamine, diolamine, glycine, lysine, meglumine, olamine, tromethamine (Tris), 2-amino-2-methylpropan-1-ol, benbthamine, erbumine (tert-butylamine), epolamine (hydroxyethylpyrrolidine), ethylenediamine, hydrabamine, morpholine, piperazine, procaine, silver, trolamine, zinc, adenine, arginine, cytosine, glucosamine, guanidine, guanine, nicotinamide, ornithine, praline, pyridoxine, serine, tyrosine, and valine. Hemisalts, for example, hemicalcium may also be formed.

Examples of pharmaceutically acceptable, non-toxic esters of the compounds of the invention include C₁-C₆ alkyl esters wherein the alkyl group is a linear or branched chain. Acceptable esters also include C₅-C₇ cycloalkyl esters as well as aralkyl esters such as, but not limited to, benzyl. C₁-C₄ alkyl esters are preferred. Esters of the compounds of the present invention may be prepared according to conventional methods.

Examples of pharmaceutically acceptable, non-toxic amides of the compounds of the invention include amides derived from ammonia, primary (C₁-C₆)alkyl amines and secondary di-(C₁-C₆)alkyl amines wherein the alkyl groups are linear or branched chain. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The use of prodrugs is contemplated by the present invention. “Prodrugs” are intended to include any covalently bonded carrier which releases the active parent drug according to Formula 1, 2, or 3, in vivo. Further, the term “prodrug” refers to compounds that are transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include acetates, formates, benzoate derivatives of alcohols, and amines present in compounds of Formula 1, 2, or 3.

The compounds of the present invention are suitable to be administered to a patient or subject for the treatment of hyperlipidemia, hypercholesterolemia, hypertriglyceridemia, and atherosclerosis. The compounds of the present invention can be administered to a patient/subject alone, or with another compound of the invention, or as part of a pharmaceutical composition.

A pharmaceutical composition of the invention contains at least one compound of the invention and at least one pharmaceutically acceptable carrier, diluent, solvent or vehicle. The pharmaceutically acceptable carrier, diluent, solvent or vehicle may be any such carrier known in the art including those described in, for example, Remington's Pharmaceutical Sciences, Mack Publishing Co., (A. R. Gennaro edit. 1985). A pharmaceutical composition of the invention may be prepared by conventional means known in the art including, for example, mixing at least one compound of the invention with a pharmaceutically acceptable carrier.

The compounds, compositions, and treatments of the present invention can be administered by any suitable means which produce contact of these compounds with the site of action in the body, for example, in the plasma, liver, rectum, or small intestine of an animal or mammal. Compositions of compounds of the invention are contemplated herein. A composition of the invention can be administered to a patient/subject either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain additives such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one (a) inert customary excipient (or carrier) such as sodium citrate or dicalcium phosphate; (b) fillers or extenders, as for example, starches, lactose, sucrose, glucose, mannitol, and silicic acid; (c) binders, as for example, carboxymethylcellulose, alignates, gelatin, polyvinylpyrrolidone, sucrose, and acacia; (d) humectants, as for example, glycerol; (e) disintegrating agents, as for example, agar-agar, calcium carbonate, potato or tapioca starch, alginic acid, certain complex silicates, and sodium carbonate; (f) solution retarders, as for example paraffin; (g) absorption accelerators, as for example, quaternary ammonium compounds; (h) wetting agents, as for example, cetyl alcohol and glycerol monostearate; (i) adsorbents, as for example, kaolin and bentonite; and (j) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. Besides inert diluents, compositions include additives, such as, for example, wetting agents, emulsifying and the pending agents, sweetening, flavoring, and perfuming agents, or mixtures thereof. Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary from patient to patient. For example, the dosage can depend on a numbers of factors including the requirements of the patient, the severity of the condition being treated, and the pharmacological activity of the compound being used. The determination of optimum dosages for a particular patient is well known to those skilled in the art.

The term “treating” or “treatment” refers to curative, palliative and prophylactic treatment, including reversing, ameliorating, alleviating, inhibiting the progress of, or preventing the disorder or condition to which such term applies, or one or more symptoms of such disorder or condition.

### Combination Aspect of the Invention

The compounds of this invention may be used, either alone or in combination with the other pharmaceutical agents described herein, in the treatment of the following diseases/conditions: dyslipidemia, hypercholesterolemia, hypertriglyceridemia, atherosclerosis, peripheral vascular disease, cardiovascular disorders, angina, ischemia, cardiac ischemia, stroke, myocardial infarction, reperfusion injury, angioplastic restenosis, hypertension, diabetes and vascular complications of diabetes, obesity, unstable angina pectoris, Alzheimer's Disease, BPH, osteoporosis, cerebrovascular disease, coronary artery disease, ventricular dysfunction, cardiac arrhythmia, pulmonary vascular disease, renal-vascular disease, renal disease, vascular hemostatic disease, autoimmune disorders, pulmonary disease, anti-oxidant disease, sexual dysfunction, cognitive dysfunction, cancer, organ transplant rejection, psoriasis, endometriosis, and macular degeneration.

The compounds of this invention may also be used in conjunction with other pharmaceutical agents (e.g., HDL-cholesterol raising agents, triglyceride lowering agents) for the treatment of the disease/conditions described herein. A combination aspect of this invention includes a pharmaceutical composition comprising a compound of this invention or its pharmaceutically acceptable salt and at least one other compound. For example, the compounds of this invention may be used in combination with cholesterol absorption inhibitors, MTP/Apo B secretion inhibitors, or other cholesterol modulating agents such as fibrates, niacin, ion-exchange resins, antioxidants, ACAT inhibitors, PPAR-activators, CETP inhibitors or bile acid sequestrants. In combination therapy treatment, both the compounds of this invention and the other drug therapies are administered to mammals by conventional methods. The following discussion more specifically describes the various combination aspects of this invention.

Any cholesterol absorption inhibitor can be used in a combination aspect of this invention. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™.

Any cholesterol ester transfer protein (“CETP”) inhibitor may be used in a combination aspect of this invention. The effect of a CETP inhibitor on lipoprotein profile is believed to be anti-atherogenic. Such inhibition is readily determined by those skilled in the art by determining the amount of agent required to alter plasma lipid levels, for example HDL cholesterol levels, LDL cholesterol levels, VLDL cholesterol levels or triglycerides, in the plasma of certain mammals, (e.g., Crook et al. Arteriosclerosis 10, 625, 1990; U.S. Pat. No. 6,140,343). A variety of these compounds are described and referenced below, however other CETP inhibitors will be known to those skilled in the art. For example, U.S. Pat. Nos. 6,197,786, 6,723,752 and 6,723,753 (the disclosures of each of which is incorporated herein by reference) disclose cholesteryl ester transfer protein inhibitors, pharmaceutical compositions containing such inhibitors and the use of such inhibitors to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascular diseases in some mammals, including humans. Examples of useful CETP inhibitors include the following compounds: [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as Torcetrapib™, and 3-{[3-(4-Chloro-3-ethyl-phenoxy)-phenyl]-[3-(1,1,2,2-tetrafluoro-ethoxy)-benzyl]-amino}-1,1,1-trifluoro-propan-2-ol. Many of the CETP inhibitors of this invention are poorly soluble and a dosage form that increases solubility facilitates the administration of such compounds. One such dosage form is a dosage form comprising (1) a solid amorphous dispersion comprising a cholesteryl ester transfer protein (CETP) inhibitor and an acidic concentration-enhancing polymer; and (2) an acid-sensitive HMG-CoA reductase inhibitor. This dosage form is more fully described in U.S. Ser. No. 10/739,567 and entitled “Dosage Forms Comprising a CETP Inhibitor and an HMG-CoA Reductase Inhibitor”, the specification of which is incorporated herein by reference.

Any compound that activates or otherwise interacts with a human peroxisome proliferator activated receptor (“PPAR”) may be used in a combination aspect of this invention. Three mammalian peroxisome proliferator-activated receptors have been isolated and termed PPAR-alpha, PPAR-gamma, and PPAR-beta (also known as NUC1 or PPAR-delta). PPAR-gamma receptors are associated with regulation of insulin sensitivity and blood glucose levels. PPAR-α activators are associated with lowering plasma triglycerides and LDL cholesterol. PPAR-β activators have been reported to both increase HDL-C levels and to decrease LDL-C levels. Thus, activation of PPAR-β alone, or in combination with the simultaneous activation of PPAR-α and/or PPAR-gamma may be desirable in formulating a treatment for dyslipidemia in which HDL is increased and LDL lowered. PPAR-activation is readily determined by those skilled in the art by the standard assays (e.g. US 2003/0225158 and US 2004/0157885). A variety of these compounds are described and referenced below, however other PPAR-activator compounds will be known to those skilled in the art. The following patents and published patent applications, the disclosure of each of which is incorporated herein by reference, provides a sampling. US 2003/0225158 discloses compounds that alter PPAR activity and methods of using them as therapeutic agents for treating or preventing dyslipidemia, hypercholesterolemia, obesity, hyperglycemia, atherosclerosis and hypertriglyceridemia. U.S. Pat. No. 6,710,063 discloses selective activators of PPAR delta. US 2003/0171377 discloses certain PPAR-activator compounds that are useful as anti-diabetic agents. US 2004/0157885 relates to PPAR agonists, in particular, certain PPARα agonists, pharmaceutical compositions containing such agonists and the use of such agonists to treat atherosclerosis, hypercholesterolemia, hypertriglyceridemia, diabetes, obesity, osteoporosis and Syndrome X or metabolic syndrome.

Examples of useful PPAR-activator compounds include the following compounds:
[5-Methoxy-2-methyl-4-(4′-trifluoromethyl-biphenyl-4ylmethylsulfanyl)-phenoxy]-acetic acid; [5-Methoxy-2-methyl-4-(3′-trifluoromethyl-biphenyl-4-ylmethylsulfanyl)-phenoxy]-acetic acid;
[4-(4′Fluoro-biphenyl-4-ylmethylsulfanyl)-5-methoxy-2methyl-phenoxy]-acetic acid; {5-Methoxy-2methyl-4-[4-(4-trifluoromethyl-benzyloxy)-benzyasulfanyl]-phenoxy}-acetic acid;
{{5-Methoxy-2-methyl-4-[4-(5-trifluoromethyl-pyridin-2-yl)-benzylsulfanyl]-phenoxy}-acetic acid;
(4-{4-[2-(3-Fluoro-phenyl)-vinyl]-benzylsulfanyl}-5-methoxy-2-methyl-phenoxy)-acetic acid; [5-Methoxy-2-methyl-4-(3-methyl-4′-trifluoromethyl-biphenyl-4-ylmethylsulfanyl)-phenoxy]-acetic acid; [5-Methoxy-2-methyl-4-(4′-trifluoromethyl-biphenyl-3-ylmethylsulfanyl)-phenoxy]-acetic acid;
{5-Methoxy-2-methyl-4-[2-(4-trifluoromethyl-benzyloxy)-benzylsulfanyl]-phenoxy}acetic acid; 3-{5-[2-(-5-Methyl-2 phenyl-oxazol-4-yl-ethoxy]-indol-1-yl}-propionic acid; 3-{4[2-(5-methyl-2-phenyl-1,3-oxazol-4-yl)ethoxy-1H-indazol-1 yl}propanoic acid; 2-Methyl-2-{3-[({2-(5-methyl-2-phenyl-1,3-oxazol-4-yl)ethoxy]carbonyl}amino)methyl]phenoxy}propionic acid; 1-{3′-[2-5-Methyl-2-phenyl-1,3-oxazol-4-y]-1,1′-biphenyl-3-yl}oxy)cyclobutanecarboxylic acid;
3-[3-(1-Carboxy-1-methyl-ethoxy)-phenyl]-piperidine-1-carboxylic acid 3-trifluoromethyl-benzyl ester;
2-{2-methyl-4-[({4-methyl-2-[4-(trifluoromethyl)phenyl]-1,3-thiazol-5-yl}methyl)sulfanyl]phenoxy}acetic acid;
2-{2-methyl-4-[({4-methyl-2-[4-(trifluoromethyl)phenyl]-1,3-oxazol-5-yl}methyl)sulfanyl]phenoxy}acetic acid;
methyl 2-{4-[({4-methyl-2-[4-(trifluoromethyl)phenyl]-1,3-thiazol-5-yl}methyl)sulfanyl]phenoxy}acetate;
2-{4-[({4-methyl-2-[4-(trifluoromethyl)phenyl]-1,3-thiazol-5-yl}methyl)sulfanyl]phenoxy}acetic acid;
(E)-3-[2-methyl-4-({4-methyl-2-[4-(trifluoromethyl)phenyl]-1,3-thiazol-5-yl }methoxy)phenyl]-2-propenoic acid;
2-{3-chloro-4-[({4-methyl-2-[4-(trifluoromethyl)phenyl]-1,3-thiazol-5-yl}methyl)sulfanyl]phenyl}acetic acid;
2-{2-methyl-4-[({4-methyl-2-[3-fluoro-4-(trifluoromethyl)phenyl]-1,3-thiazol I-5-yl}methyl)sulfanyl]phenoxy}acetic acid; and pharmaceutically acceptable salts thereof.

Any MTP/Apo B secretion (microsomal triglyceride transfer protein and/or apolipoprotein B secretion) inhibitor can be used in the combination aspect of the present invention. Such inhibition is readily determined by those skilled in the art according to standard assays (e.g., Wetterau, J. R. 1992; Science 258:999). A variety of these compounds are known to those skilled in the art, including imputapride (Bayer) and additional compounds such as those disclosed in WO 96/40640 and WO 98/23593.

Any ACAT inhibitor can serve in the combination therapy aspect of the present invention. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor can serve in the combination therapy aspect of the present invention. Such lipase inhibition activity is readily determined by those skilled in the art according to standard assays (e.g., Methods Enzymol. 286: 190-231). Pancreatic lipase mediates the metabolic cleavage of fatty acids from triglycerides at the 1- and 3-carbon positions. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231). Gastric lipase is an immunologically distinct lipase that is responsible for approximately 10 to 40% of the digestion of dietary fats. Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949). Lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexa-decanoic 1,3 acid lactone, and the variously substituted N-formylleucine derivatives and stereoisomers thereof, are disclosed in U.S. Pat. No. 4,598,089. Tetrahydrolipstatin may be prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[-(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpipe-ridin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Bile acid sequestrants, such as Welchol®, Colestid®, LoCholest®, Questran® and fibric acid derivatives, such as Atromid®, Lopid® and Tricor® may be used in a combination aspect of the invention.

Compounds of the present invention can be used with anti-diabetic compounds. Diabetes can be treated by administering to a patient having diabetes (especially Type II), insulin resistance, impaired glucose tolerance, or the like, or any of the diabetic complications such as neuropathy, nephropathy, retinopathy or cataracts, a therapeutically effective amount of a Formula I compound in combination with other agents (e.g., insulin) that can be used to treat diabetes. This includes the classes of anti-diabetic agents (and specific agents) described herein.

Any glycogen phosphorylase inhibitor can be used in combination with a Formula I compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a Formula I compound of the present invention. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a compound of the present invention. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a Formula I compound of the present invention. Such glucosidase inhibition activity is readily determined by those skilled in the art according to standard assays (e.g., Biochemistry (1969) δ: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydr-oxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihyd-roxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-.beta.-D-glucopyranosyl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-.alpha.-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, may be used in combination with a Formula I compound of the present invention, includes, for example, the following: biguanides (e.g., mefformin), insulin secretagogues (e.g., sulfonylureas and glinides), glitazones, non-glitazone PPAR gamma agonists, PPAR.beta. agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-.beta. inhibitors and AGE breakers.

Compounds of the present invention can be used in combination with anti-obesity agents. Any anti-obesity agent can be used in such combinations and examples are provided herein. Such anti-obesity activity is readily determined by those skilled in the art according to standard assays known in the art. Suitable anti-obesity agents include phenylpropanolamine, ephedrine, pseudoephedrine, phentermine, beta sub.3 adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor antagonists (e.g., rimonabant (SR-141,716A)), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Any thyromimetic can be used in combination with compounds of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629. and bromocriptine which can be prepared as described in U.S. Pat. Nos. 3,752,814 and 3,752,888.

Anti-resorptive agents (for example progestins, polyphosphonates, bisphosphonate(s), estrogen agonists/antagonists, estrogen, estrogen/progestin combinations, Premarin®, estrone, estriol or 17.alpha.- or 17.beta.-ethynyl estradiol) may be used in conjunction with the compounds of Formula I of the present invention. Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, uetonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol. Preferred progestins are medroxyprogestrone, norethindrone and norethynodrel. Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl)amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

The compounds of this invention may be combined with a mammalian estrogen agonist/antagonist. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods (Eriksen E. F. et al., Bone Histomorphometry, Raven Press, New York, 1994, pages 1-74; Grier S. J. et. al., The Use of Dual-Energy X-Ray Absorptiometry In Animals, “Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911. Another preferred estrogen agonist/antagonist is tamoxifen: (ethanamine,2-(-4-(1,2-diphenyl-1-butenyl)phenoxy)-N,N-dimethyl, (Z)-2-, 2-hydroxy-1,2,3-propanetricarboxylate (1:1)) and related compounds which are disclosed in U.S. Pat. No. 4,536,516, the disclosure of which is incorporated herein by reference. Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)eth-oxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference. Another preferred estrogen agonist/antagonist is centchroman: 1-(2-((4-(-methoxy-2,2,dimethyl-3-phenyl-chroman-4-yl)-phenoxy)-ethyl)-p-pyrrolidine, which is disclosed in U.S. Pat. No. 3,822,287, the disclosure of which is incorporated herein by reference. Also preferred is levormeloxifene. Another preferred estrogen agonist/antagonist is idoxifene: (E)-1-(2-(4-(1-(4-iodo-phenyl)-2-phenyl-but-1-enyl)-phenoxy)-ethyl)-pyrro-lidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thio-phen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hyd-roxy-phenyl)-benzo[b]thiophen-3-yl)-methanone which is disclosed, along with methods of preparation, in PCT publication no. WO 95/10513 assigned to Pfizer Inc., the disclosure of which is incorporated herein by reference.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:
cis-6-(4-fluoro-phenyl)-5-(4-(2-piperidin-1-yl-ethoxy)-phenyl)-5,6,-7,8-tetrahydronaphthalene-2-ol;
(−)-cis-6-phenyl-5-(4-(2-pyrrolidin-1-yl-ethoxy)-phenyl)-5,6,7,8-te-trahydro-naphthalene-2-ol (also known as lasofoxifene);
cis-6-phenyl-5-(4-(2-pyrrolidin-1-yl-ethoxy)-phenyl)-5,6,7,8-tetrah-ydro-naphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,-4-tetrahydroisoquinoline;
is-6-(4-hydroxyphenyl)-5-(4-(2-piperidin-1-yl-ethoxy)-phenyl)-5,6,-7,8-tetrahydronaphthalene-2-ol; and
1-(4′-pyrrolidinolethoxyphenyl)-2-phenyl-6-hydroxy-1,2,3,4-tetrahyd-roisoquinoline.
   Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzoth-iophene and 2-phenyl-3-aroylbenzothiophene-1-oxide.

Other anti-osteoporosis agents, which can be used in combination with a Formula I compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any compound that is an antihypertensive agent may be used in a combination aspect of this invention. Such compounds include amlodipine and related dihydropyridine compounds, calcium channel blockers, angiotensin converting enzyme inhibitors (“ACE-Inhibitors”), angiotensin-II receptor antagonists, beta-adrenergic receptor blockers and alpha-adrenergic receptor blockers. Such antihypertensive activity is determined by those skilled in the art according to standard tests (e.g. blood pressure measurements).

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate and other pharmaceutically acceptable acid addition salts of amlodipine have utility as antihypertensive agents and as antiischemic agents. Amlodipine and its pharmaceutically acceptable acid addition salts are also disclosed in U.S. Pat. No. 5,155,120 as having utility in the treatment of congestive heart failure. Amlodipine besylate is currently sold as Norvasc®.

Calcium channel blockers which are within the scope of a combination aspect of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, prenylamine, semotiadil, terodiline, verapamil, aranipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline The disclosures of all such U.S. patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, ceronapril, delapril, enalapril, fosinopril, imadapril, lisinopril, moveltopril, perindopril, quinapril, ramipril, spirapril, temocapril, and trandolapril. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, losartan, and valsartan. The disclosures of all such U.S. patents are incorporated herein by reference.

Beta-adrenergic receptor blockers (beta- or. beta.-blockers) which are within the scope of this invention include, but are not limited to: acebutolol, which may be prepared as disclosed in U.S. Pat. No. 3,857,952; alprenolol, amosulalol, which may be prepared as disclosed in U.S. Pat. No. 4,217,305; arotinolol, atenolol, befunolol, betaxolol; The disclosures of all such U.S. patents are incorporated herein by reference.

Alpha-adrenergic receptor blockers (alpha- or .alpha.-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, doxazosin, fenspiride, indoramin, labetolol, naftopidil, nicergoline, prazosin, tamsulosin, tolazoline, trimazosin, and yohimbine, which may be isolated from natural sources according to methods well known to those skilled in the art. The disclosures of all such U.S. patents are incorporated herein by reference.

Any compound that is known to be useful in the treatment of Alzheimer's Disease may be used in a combination aspect of this invention. Such compounds include acetylcholine esterase inhibitors. Examples of known acetylcholine esterase inhibitors include donepezil (Aricept®), tacrine (Cognex®), rivastigmine (Exelon®) and galantamine (Reminyl). Aricept® is disclosed in the following U.S. patents, all of which are fully incorporated herein by reference: U.S. Pat. Nos. 4,895,841, 5,985,864, 6,140,321, 6,245,911 and 6,372,760. Exelon® is disclosed in U.S. Pat. Nos. 4,948,807 and 5,602,176 which are fully incorporated herein by reference. Cognex® is disclosed in U.S. Pat. Nos. 4,631,286 and 4,816,456 (fully incorporated herein by reference). Remynil® is disclosed in U.S. Pat. Nos. 4,663,318 and 6,099,863 which are fully incorporated herein by reference.

### Preparation of the Compounds of the Invention

The present invention contains compounds that can be synthesized in a number of ways familiar to one skilled in organic synthesis. The compounds outlined herein can be synthesized according to the methods described below, along with methods typically used by a synthetic organic chemist, and combinations or variations of those methods, which are generally known to one skilled in the art of synthetic chemistry. The synthetic route of compounds in the present invention is not limited to the methods outlined below. One skilled in the art will be able to use the schemes below to synthesize compounds claimed in this invention. Individual compounds may require manipulation of the conditions in order to accommodate various functional groups. A variety of protecting groups known to one skilled in the art may be required. Purification, if necessary, may be accomplished on a silica gel column eluted with the appropriate organic solvent system. Also, reverse phase HPLC or recrystallization may be employed. The following non-limiting descriptions also demonstrate methods for the synthesis of compounds of the invention.

A synthesis of 1-[2-((4R,6R)-6-tert-Butoxycarbonylmethyl-2,2-dimethyl [1,3]dioxan-4-yl)-ethyl]-2-(4-fluoro-phenyl)-5-cyclopropyl-1H-imidazole-4-carboxylic acid 5 is illustrated in Scheme 1. Thus, commercially available (benzhydrylidene-amino)-acetic acid benzyl ester 1 (Bridge Organics) is acylated with cyclopropanecarbonyl chloride according to the method of J. Singh et al (Tetrahedron Lett. 1993, 34, 211). Subsequent acid hydrolysis provides the amine 2. A second acylation is accomplished by reacting compound 2 with p-fluorobenzoyl chloride under basic condition to give 3. Condensation-cyclodehydration of 3 with [(4R,6R)-6-(2-Amino-ethyl)-2,2-dimethyl-[1,3]dioxan-4-yl]-acetic acid tert-butyl ester yields the benzyl ester 4. Hydrogenolysis of 4 yields the free acid 5.

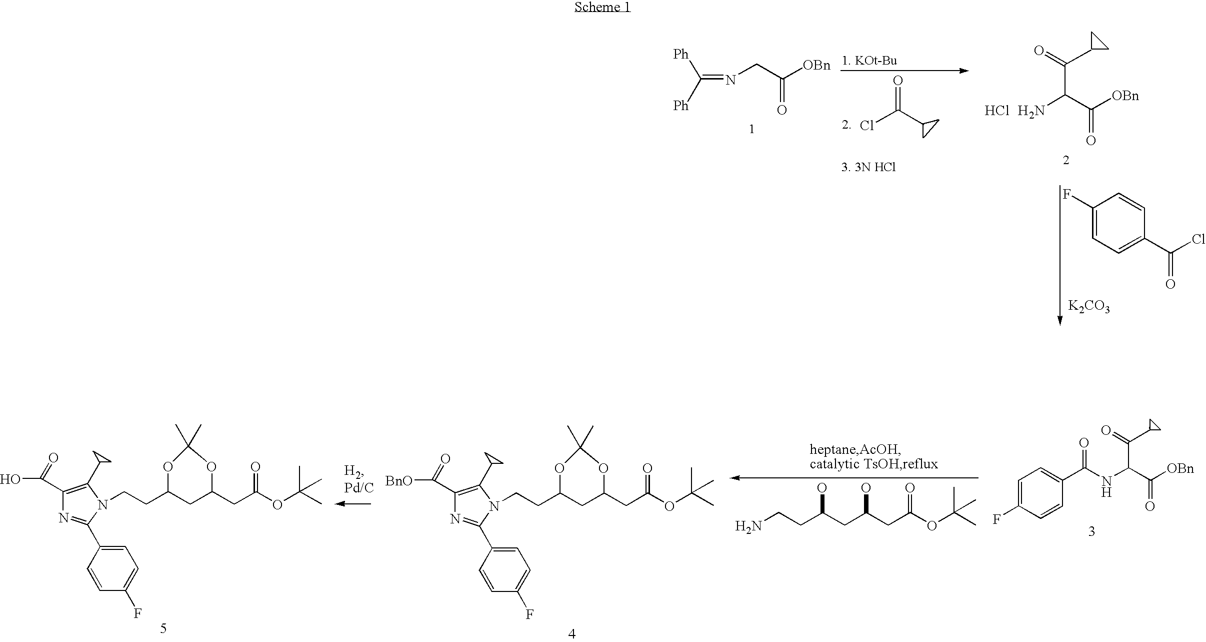

Scheme 2 illustrates a method for preparation of compounds of the invention from the carboxylic acid 5. Thus, in situ activation of 5 with EDCl/HOBt, or a similar activating agent, and treatment with, for example, (R)-1-Phenyl-ethylamine gives the amide 6. Exposure of 6 to TFA provides the lactone 7 which is converted to 8 on treatment with base. The crude coupling product 6 may be converted directly to the Lactone 7 without isolation.

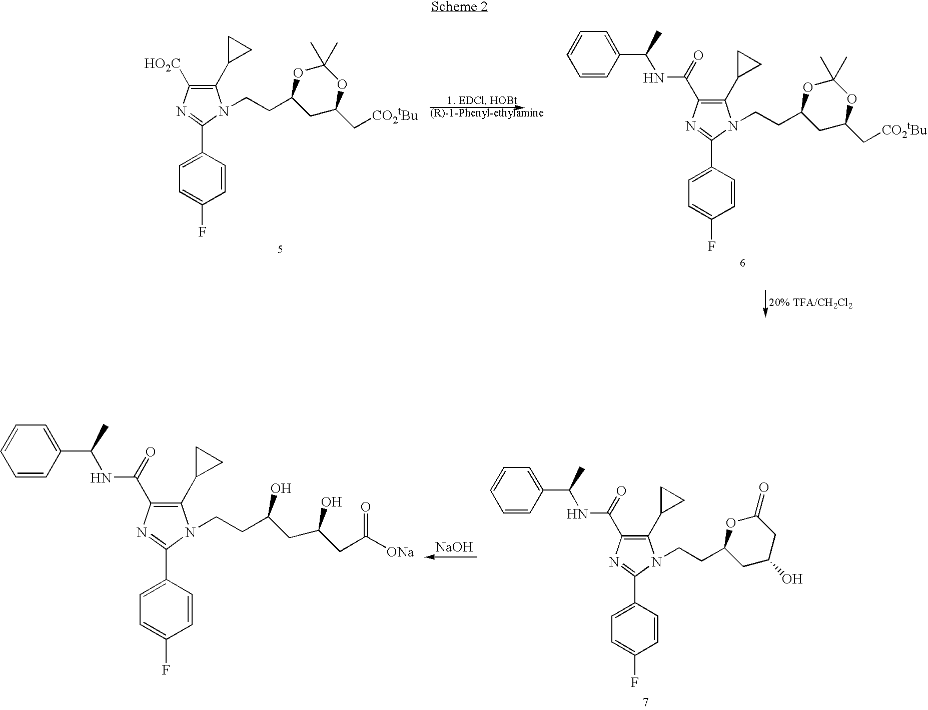

Scheme 3 illustrates a further method for preparation of compounds of the invention from the carboxylic acid 5. Thus, in situ activation of 5 with EDCl/HOBt, or a similar activating agent, and treatment with, for example, (S)-1-Phenyl-ethylamine gives the amide 9. Exposure of 9 to methanolic HCl provides the diol 10 which is converted to 11 on treatment with base.

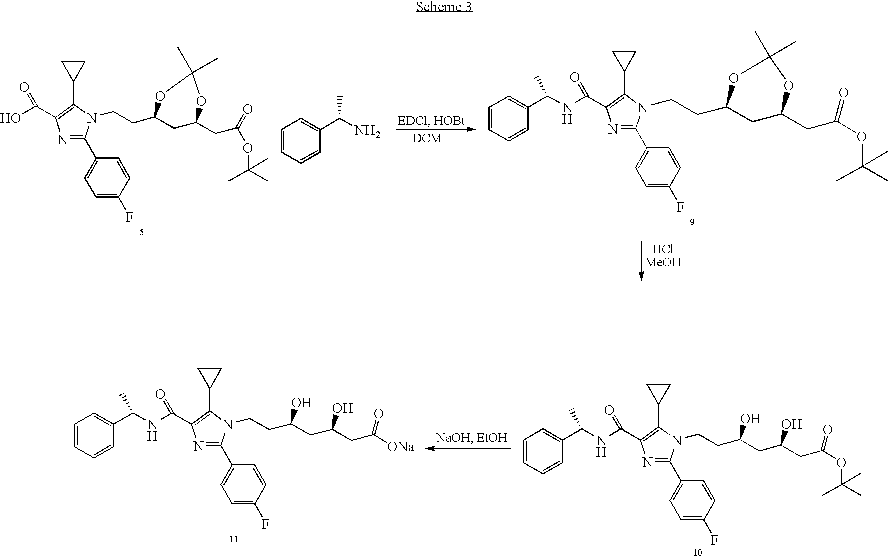

Scheme 4 illustrates a method for the preparation of, for example, imidazole sodium salt 16 from ketoamide 3. Thus, aminolysis of benzylester 3 with benzylamine yields benzylamide 12. Treatment of 12 with TBIA and benzoic acid or phenylacetic acid in refluxing heptane affords imidazole 13. Acid-catalyzed removal of the acetal yields diol 14, and subsequent hydroxide saponification, followed by acid-catalyzed cyclodehydration affords lactone 15. Lactone 15 is converted to imidazole sodium salt 16 by treatment with aqueous sodium hydroxide. Alternatively, treatment of diol 14 with NaOH will give 16 directly. Recrystallization of crude sodium salt 16 affords material of high purity.

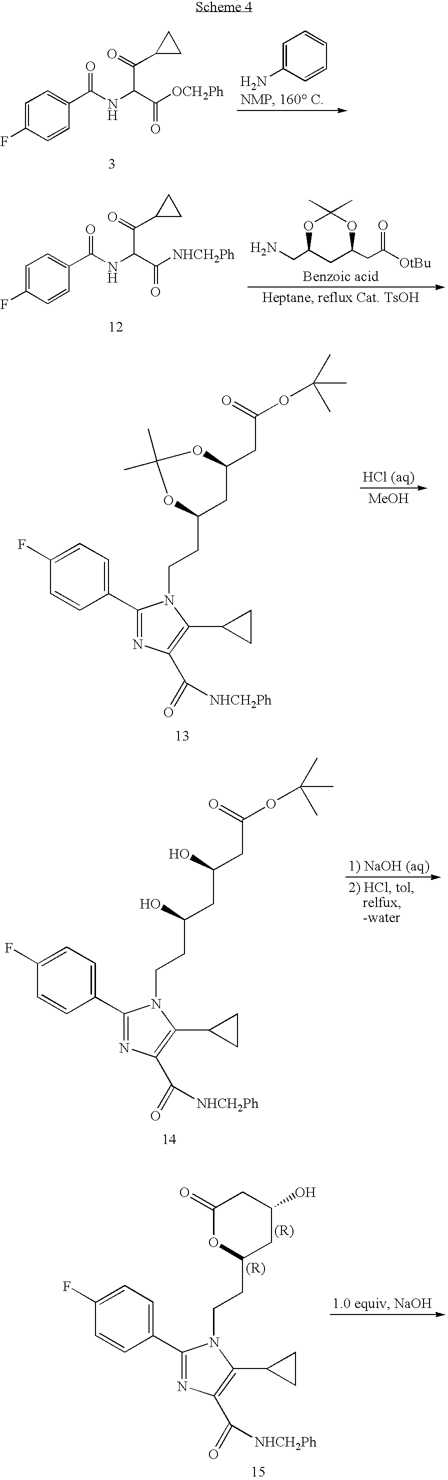

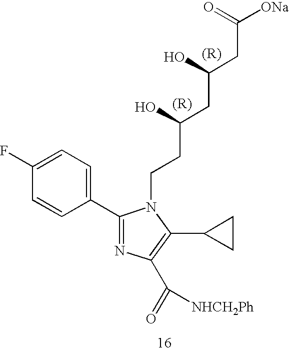

### EXAMPLES

The following non-limiting Examples show how to carry out the present invention. The synthetic route to compounds of the present invention is not limited to the methods outlined below. One skilled in the art will be able to use the schemes outlined above to synthesize various compounds claimed in this invention.

### Example 1

### Sodium: (3R,5R)-7-[5-Cyclopropyl-2-(4-fluoro-phenyl)-4-((R)-1-Phenyl-ethylcarbamoyl)-imidazol-1-yl]-3,5-dihydroxy-heptanoate

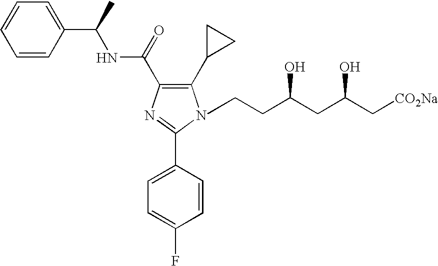 Step A

### 3-Cyclopropyl-2-(4-fluoro-benzoylamino)-3-oxo-propionic acid benzyl ester

A 500 mL round-bottomed flask was charged with potassium tert-butoxide (9.4 g, 83 mmol) and THF (150 mL). The solution was cooled, under nitrogen, in an ice-brine bath and treated with a solution of (Benzhydrylidene-amino)-acetic acid benzyl ester (25.0 g, 79.5 mmol) in THF (150 mL). The red-orange solution was stirred for 1 h at 0° C. and then cannulated into a −78° C. solution of cyclopropanecarbonyl chloride (8.33 g, 79.7 mmol) in THF (400 mL). The resulting mixture was stirred for 2 h at −78° C., then quenched with 3M HCl (75 mL, 225 mmol). The cold bath was removed and the reaction mixture was allowed to stand overnight. The reaction mixture was concentrated in vacuo to produce an oily yellow residue, low resolution mass spectroscopy (APCI) m/z 232[M−H]⁻. The crude oil was dissolved in water (200 mL) and extracted with ether (2×100 mL). The aqueous layer was adjusted to pH >8 by the careful addition of solid NaHCO₃. EtOAc was added (300 mL), the biphasic mixture was cooled in an ice-brine bath, and the cooled mixture was treated with 4-fluorobenzoyl chloride (12.6 g, 79.7 mmol). The reaction mixture was allowed to warm to rt and left to stand overnight. The organic layer was separated, washed sequentially with 1 M HCl and sat. NH₄Cl, dried (Na₂SO₄), and concentrated to a crude oil that solidified on standing. The crude product was recrystallized from a minimum of hot 95% EtOH to give colorless needles that were collected by vacuum filtration. The purified material was dried in vacuo. Yield: 14.2 g (52%); mp=94.5-96° C.; Low resolution mass spectroscopy (APCI) m/z 354[M−H]⁻; Anal. Calcd. For C₂₀H₁₈F₁N₁O₄. Theory: C, 67.67; H, 5.11; N, 3.94. Found: C, 67.48; H, 5.12; N, 3.90.

Step B

### 1-[2-((4R,6R)-6-tert-Butoxycarbonylmethyl-2,2-dimethyl-[1.3]dioxan-4-yl)-ethyl]-2-(4-fluoro-phenyl)-5-cyclopropyl-1H-imidazole-4-carboxylic acid benzyl ester

A mixture of 3-Cyclopropyl-2-(4-fluoro-benzoylamino)-3-oxo-propionic acid benzyl ester (6.0 g, 17 mmol), [(4R,6R)-6-(2-Amino-ethyl)-2,2-dimethyl-[1,3]dioxan-4-yl]-acetic acid tert-butyl ester (TBIA) (9.2 g, 33.8 mmol), benzoic acid (6.19 g, 50.7 mmol), and p-toluenesulfonic acid (0.29 g, 1.7 mmol) in n-heptane (150 mL) was heated to reflux for 65 h with the removal of water (Dean-Stark trap). The reaction mixture was cooled, diluted with EtOAc (100 mL), and washed with 1M NaOH (2×150 mL) and sat NH₄Cl, dried (Na₂SO₄) and concentrated to a yellow-brown oil. Purification by flash chromatography [SiO₂, Ethyl Acetate/hexanes 10-50%] provides the desired product as a yellow glass that was dried under high vacuum. Yield: 2.1 g (21%); Low resolution mass spectroscopy (APCI) m/z 593 [M+H]⁺; Anal. Calcd. For C₃₄H₄₁F₁N₂O₆: C, 68.90; H, 6.97; N, 4.73. Found: C, 68.66; H, 7.01; N, 4.64.

Step C

### 1-[2-((4R,6R)-6-tert-Butoxycarbonylmethyl-2,2-dimethyl-[1.3]dioxan-4-yl)-ethyl]-5-cyclopropyl-2-(4-fluoro-phenyl)-1H-imidazole-4-carboxylic acid

A solution of 1-[2-((4R,6R)-6-tert-Butoxycarbonylmethyl-2,2-dimethyl-[1,3]dioxan-4-yl)-ethyl]-2-(4-fluoro-phenyl)-5-cyclopropyl-1H-imidazole-4-carboxylic acid benzyl ester (2.0 g, 3.4 mmol) in THF (200 mL) was hydrogenated over 20% Pd/C until the uptake of hydrogen ceased. The solution was filtered through celite and concentrated to give the title compound as a colorless foam; yield: 1.69 g (99%); Low resolution mass spectroscopy (APCI) m/z 503 [M+H]⁺; Anal. Calcd. For C₂₇H₃₅F₁N₂O₆: C, 64.53; H, 7.02; N, 5.57. Found: C, 63.99; H, 7.38; N, 5.25.

Step D

### 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid ((R)-1-phenyl-ethyl)-amide

A rt solution 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (840 mg, 1.67 mmol) in dry DMF (35 mL) was treated with EDCl (380 mg, 2.0 mmol) and HOBt monohydrate (330 mg, 2.2 mmol). After stirring for 20 min, neat (R)-1-phenylethylamine (640 mg, 5.28 mmol) was added and the reaction was allowed to stir at rt overnight. An LC-MS analysis of the crude reaction mixture indicates a mass corresponding to the expected product [M+H]⁺=606. The reaction mixture was poured into water (150 mL) and extracted with EtOAc (3×). The extracts were combined, washed with water (2×) and sat. NH₄Cl (2×), dried (Na₂SO₄) and concentrated to a colorless foam. The crude amide was taken up in CH₂Cl₂ (20 mL), treated with neat TFA (5 mL), and allowed to stir at rt for 30 min at which time an LC-MS analysis indicated no remaining SM and a new mass corresponding to the expected lactone [M+H]⁺=492. The reaction mixture was concentrated to dryness and residue was partitioned between EtOAc and 1 M NaHCO₃. (pH˜8). The organic layer was separated, washed with sat. NH₄Cl, dried (Na₂SO₄), and concentrated to an oil. Purification by flash chromatography (silica, EtOAc/hexanes 50-100%) provides the lactone as a colorless foam. Yield: 482 mg (57%); Low resolution mass spectroscopy (APCI) m/z 492 [M+H]⁺; Anal. Calcd. For C₂₈H₃₀F₁N₃O₄/0.11 C₄H₈O₂ (EtOAc): C, 68.15; H, 6.21; N, 8.38. Found: C, 67.78; H, 6.10; N, 8.30.

Step E

A solution of the 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid ((R)-1-phenyl-ethyl)-amide (395 mg, 0.80 mmol) in THF (4 mL) was treated with aqueous NaOH (5.44 mL, 0.147 M, 1.00 eq). The reaction mixture was allowed to stir until an analysis of the reaction mixture by loop injection LC-MS indicated that the starting material was consumed. The sample was concentrated to approximately 4.0 mL, diluted with water (25 mL) and lyophilized to give a colorless powder; Yield: 0.427 g (99%); Low resolution mass spectroscopy (APCI) m/z 510 [M+H]⁺; Anal. Calcd. For C₂₈H₃₁F₁N₃Na₁O₅/1.1H₂O: C, 60.99; H, 6.07; N, 7.62. Found: C, 61.02; H, 5.76; N, 7.49.

### Example 2

### Sodium; (3R,5R)-7-{5-Cyclopropyl-2-(4-fluoro-phenyl)-4-[methyl-((R)-1-phenyl-ethyl)-carbamoyl]-imidazol-1-yl}-3,5-dihydroxy-heptanoate

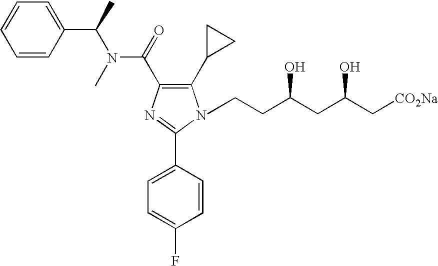 Step A

### 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid methyl-((R)-1-phenyl-ethyl)-amide

A solution of 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (1.5 g, 3.0 mmol) and 2,6-lutidine (0.695 mL, 6.0 mmol) in dry CH₃CN (35 mL) was cooled, under a nitrogen atmosphere, to approximately 0° C. (ice-water bath) and treated with pentafluorophenyl trifluoroacetate (0.615 mL, 3.6 mmol). The reaction flask was removed from the cooling bath and the reaction was allowed to stir at ambient temperature for 2 h. The reaction mixture was treated with neat methyl-((R)-1-phenyl-ethyl)-amine (0.835 mL, 6.0 mmol) and stirring was continued overnight. LC-MS analysis of the crude reaction mixture indicates a mass corresponding to the expected coupling product (APCI) m/z 620 [M+H]⁺. The reaction mixture was poured into water and extracted with EtOAc (2×). The extracts were combined, washed with water and sat. NH₄Cl, dried (Na₂SO₄) and concentrated to a yellow oil. The crude material was dissolved in CH₂Cl₂ (50 mL), cooled in an ice bath, treated with neat TFA (15 mL) and allowed to stir until an HPLC-MS analysis indicates no remaining SM and a new peak corresponding to the expected lactone (APCI) m/z 506 [M+H]⁺. The reaction was diluted with EtOAc and water. The aqueous layer was carefully neutralized with 1 M NaHCO₃. (pH˜8). The organic layer was separated, washed with sat. NH₄Cl, dried (Na₂SO₄), and concentrated to an oil. Purification by flash chromatography (silica, MeOH/EtOAc 0-10%) provides the lactone as a colorless foam which was dried in vacuo at 55° C. Yield: 0.36 g (23%); Low resolution mass spectroscopy (APCI) m/z 506 [M+H]⁺.

Step B

A solution of 5-Cyclopropyl-2-(4-fluoro-phenyl)-1-[2-((2R,4R)-4-hydroxy-6-oxotetrahydro-pyran-2-yl)-ethyl]-1H-imidazole-4-carboxylic acid methyl-((R)-1-phenyl-ethyl)-amide (178 mg, 0.35 mmol) in THF (4 mL) was treated with aqueous NaOH (2.38 mL, 0.147 M, 1.00 eq). The reaction mixture was allowed to stir at rt for 30 min. An analysis of the reaction mixture by loop injection LC-MS indicated that the starting material was consumed. The sample was concentrated to approximately 2.0 mL, diluted with water (5 mL) and lyophilized to give a colorless powder; Yield: 0.189 g (98%); Low resolution mass spectroscopy (APCI) m/z 524 [M+H]⁺; Anal. Calcd. For C₂₉H₃₃F₁N₃Na₁O₅/1.4H₂O: C, 61.02; H, 6.32; N, 7.36. Found: C, 61.07; H, 6.00; N, 7.20.

### Example 3

### Sodium; (3R,5R)-7-[5-Cyclopropyl-2-(4-fluoro-phenyl)-4-((S)-1-phenyl-ethylcarbamoyl)-imidazol-1-yl]-3,5-dihydroxy-heptanoate

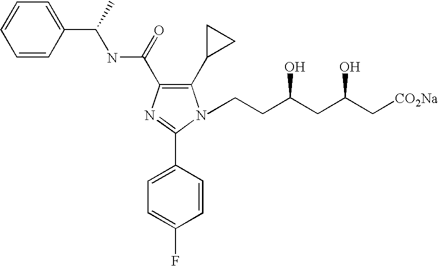 Step A

### ((4R,6R)-6-{2-[5-Cyclopropyl-2-(4-fluoro-phenyl)-4-((S)-1-phenyl-ethylcarbamoyl)-imidazol-1-yl]-ethyl}-2,2-dimethyl-[1.3]dioxan-4-yl)-acetic acid tert-butyl ester

A solution of 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (1.0 g, 2.0 mmol) in 25 mL of CH₂Cl₂ was treated with EDCl (0.57 g, 3.0 mmol) and HOBt-monohydrate (0.46 g, 3.0 mmol). The resulting mixture was allowed to stir at ambient temperature for 20 min, then treated neat (S)-methyl benzylamine (0.38 mL, 3.0 mmol). The dark orange mixture was allowed to stir at rt overnight. The solvent was removed and the crude residue was partitioned between EtOAc and water. The organic phase was separated, and the aqueous layer was extracted with EtOAc (2×). The combined organic extracts were washed with sat. NH₄Cl and brine, dried (MgSO₄), filtered and concentrated to a crude dark orange oil.

Purification by flash chromatography (silica, EtOAc/hexanes 50%) provided the amide as a yellow oil; Yield: 1.07 g (88%); Low resolution mass spectroscopy (APCI) m/z 606 [M+H]⁺.

Step B

### (3R,5R)-7-[5-Cyclopropyl-2-(4-fluoro-phenyl)-4-((S)-1-phenyl-ethylcarbamoyl)-imidazol-1-yl]-3,5-dihydroxy-heptanoic acid tert-butyl ester

A solution of ((4R,6R)-6-{2-[5-Cyclopropyl-2-(4-fluoro-phenyl)-4-((S)-1-phenyl-ethylcarbamoyl)-imidazol-1-yl]-ethyl}-2,2-dimethyl-[1,3]dioxan-4-yl)-acetic acid tert-butyl ester (0.50 g, 0.83 mmol) in 10 mL of MeOH was treated with hydrochloric acid (3.44 mL, 4.13 mmol, 1.2 M). The resulting mixture was allowed to stir at rt for 2 hrs. The yellow solution was adjusted to pH 7 by the careful addition of saturated NaHCO₃, diluted with water and extracted (2×) with CH₂Cl₂. The organic extracts organics were combined, washed with brine, dried (MgSO₄), and concentrated down to a crude orange oil. Purification by flash chromatography (silica, EtOAc/hexanes 50-100%) provided the desired product as a yellow foam. Yield: 0.34 g (72%); Low resolution mass spectroscopy (APCI) m/z 566 [M+H]⁺

Step C

### Sodium; (3R,5R)-7-[5-Cyclopropyl-2-(4-fluoro-phenyl)-4-((S)-1-phenyl-ethylcarbamoyl)-imidazol-1-yl]-3,5-dihydroxy-heptanoate

A solution of (3R,5R)-7-[5-Cyclopropyl-2-(4-fluoro-phenyl)-4-((S)-1-phenyl-ethylcarbamoyl)-imidazol-1-yl]-3,5-dihydroxy-heptanoic acid tert-butyl ester (0.29 g, 0.51 mmol) in EtOH (10 mL) was treated with aqueous NaOH (0.49 mL, 0.51 mmol, 1.04M). The clear mixture was allowed to stir until an LC-MS analysis of the reaction mixture indicated no remaining starting material. The reaction mixture was concentrated under high vacuum to a colorless foam. Yield: 0.268 g (99%); Low resolution mass spectroscopy (APCI) m/z 510 [M+H]⁺, Anal. Calcd. For C₂₈H₃₁F₁N₃Na₁O₅/2.1H₂O: C, 58.96; H, 6.40; N, 7.37. Found: C, 58.58; H, 5.91; N, 7.04.

### Formulations

The compounds of the present invention including those exemplified herein and all compounds of Formulas I-III, hereafter referred to as “compound(s)” can be administered alone or in combination with one or more therapeutic agents. These include, for example, other agents for treating, preventing or controlling dyslipidemia, non-insulin dependent diabetes mellitus, obesity, hyperglycemia, hypercholesteremia, hyperlipidemia, atherosclerosis, hypertriglyceridemia, or hyperinsulinemia.

The compounds are thus well suited to formulation for convenient administration to mammals for the prevention and treatment of such disorders.

The following examples further illustrate typical formulations of the compounds provided by the invention.

Formulation 1

| Ingredient | Amount | |
|---|---|---|
| compound | 0.5 to 800 | mg |
| sodium benzoate | 5 | mg |
| isotonic saline | 1000 | mL |

The above ingredients are mixed and dissolved in the saline for IV administration to a patient.

Formulation 2

| Ingredient | Amount | |
|---|---|---|
| compound | 0.5 to 800 | mg |
| cellulose, microcrystalline | 400 | mg |
| stearic acid | 5 | mg |
| silicon dioxide | 10 | mg |
| sugar, confectionery | 50 | mg |

The ingredients are blended to uniformity and pressed into a tablet that is well suited for oral administration to a patient.

Formulation 3

| Ingredient | Amount | |
|---|---|---|
| compound | 0.5 to 800 | mg |
| starch, dried | 250 | mg |
| magnesium stearate | 10 | mg |

The ingredients are combined and milled to afford material suitable for filling hard gelatin capsules administered to a patient.

Formulation 4

| Ingredient | Amount % wt./(total wt.) |
|---|---|
| compound | 1 to 50 |
| Polyethylene glycol 1000 | 32 to 75 |
| Polyethylene glycol 4000 | 16 to 25 |

The ingredients are combined via melting and then poured into molds containing 2.5 g total weight.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

### Biological Assays

The compounds of the invention have demonstrated HMG Co-A reductase inhibition in standard assays commonly employed by those skilled in the art. (See, e.g., J. of Lipid Research 1998; 39:75-84; Analytical Biochemistry, 1991; 196:211-214; RR 740-01077 Pharmacology 8 November 82). Accordingly, such compounds and formulations comprising such compounds are useful for treating, controlling or preventing inter alia hypercholesterolemia, hyperlipidemia, hypertriglyceridemia or atherosclerosis.

A.) In Vitro Assay

Rat Liver Microsomal Isolation Procedure:

Male Charles River Sprague-Dawley rats were fed with 2.5% cholestyramine in rat chow diets for 5 days before sacrificing. Livers were minced and homogenized in a sucrose homogenizing solution in an ice bath 10 times. Homogenates were diluted into a final volume of 200 mL, and centrifuged 15 min. with a Sorvall Centrifuge at 5° C., 10,000 rpm (12,000×G). The upper fat layer was removed and the supernatant decanted into fresh tubes. This step was repeated one more time before transferring the supernatant into ultracentrifuge tubes and centrifuged at 36,000 rpm (105,000×G) for an hour at 5° C. The resulting supernatant was discarded and the pellet was added to total of 15 mL 0.2 M KH₂PO₄. Pellets were homogenized gently by hand about 10 times. Samples were pooled and diluted into total of 60 mL buffer. The protein concentration of the homogenate was determined by the Lowry Method using a BCA (Bicinchoninic acid), kit from Pierce Chemical Company. 1 mL aliquots of microsomes were kept frozen in liquid nitrogen.

### HMGCoA (3-Hydroxy-3-methylglutaryl CoA) Reductase Assay

Materials and Methods:

[3-¹⁴C]-HMGCoA (57.0 mCi/mmol) was purchased from Amersham Biosciences, UK. HMGCoA, mevalonolactone, β-NADPH (β-Nicotinamide Adenine Dinucleotide Phosphate, Reduced form) were purchased from Sigma Chemical Co. AG 1-8X resin was purchased from Bio-Rad Laboratory.
1. One μL of dimethyl sulfoxide (DMSO) or 1 μL of DMSO containing a test compound at a concentration sufficient to give a final assay concentration of between 0.1 nM to 1 mM was placed into each well of a Corning 96 well plate. A Volume of 34 μL of buffer (100 mM NaH₂PO₄, 10 mM Imidazole and 10 mM EDTA), (Ethylenediaminetetra acetic acid) containing with 50 μg/mL rat liver microsomes was added into each well. After incubation for 30 min. on ice, 15 μL of ¹⁴C-HMGCoA (0.024 μCi) with 15 mM NADPH, 25 mM DTT, (Dithiothreitol) was added and incubated at 37° C. for an additional 45 min. The reaction was terminated by the addition of 10 μL of HCl followed by 5 μL of mevalonolactone. Plates were incubated at room temperature overnight to allow lactonization of mevalonate to mevalonolactone. The incubated samples were applied to columns containing 300 μL of AG1-X8 anion exchange resin in a Corning filter plate. The eluates were collected into Corning 96 well capture plates. Scintillation cocktail (Ultima-Flo-M) was added into each well and plates counted on a Trilux Microbeta Counter. The IC₅₀ values were calculated with GraphPad software (Prism).
   Procedure:
2. Add 1 μL DMSO or compounds into the wells according to the protocol
3. Add 35 μL incubation buffer with the rat microsomes into each well. Incubate 30 min. at 4° C.
4. Add 15 μL ¹⁴C-HMGCoA. Incubate 45 min. at 37° C.
5. Add 10 μL HCl stop reagent
6. Add 5 μL mevelonolactone. Incubate overnight at room temperature
7. Apply the containing into the AG 1-X8 anion exchange resin in Corning filter plate
8. Collect the eluate into Corning capture plate
9. Add scintillation cocktail Ultima-Flo-M
10. Count on a Trilux Microbeta Counter μ
11. Calculate IC₅₀ values

Compounds of the invention exhibit a range of IC₅₀ values of less than about 600 nM in the aforementioned in vitro assay. See, for example, the compounds of: Example 2, which has an IC₅₀ of 36.8 nM.

B.) Cell Assay

Protocol for Sterol Biosynthesis in Rat Hepatocytes:

Cell Culture, Compounds Treatment and Cell Labeling:

Frozen rat hepatocytes purchased from XenoTech (cat# N400572) were seeded on 6-well collagen I coated plates at a density of 10⁵ cells/per well. The cells were grown in DMEM, (Dulbecco's Modified Eagle Medium) (Gibco, #11054-020) containing 10% FBS (Fetal Bovine Serum) and 10 mM HEPES, (N-2-hydroxyethyl-piperazine-N¹-2-ethane sulfonic acid) (Gibco # 15630-080) for 24 hrs. The cells were pre-incubated with compounds for 4 hrs and then labeled by incubating in medium containing 1 μCi/per mL of ¹⁴C acetic acid for an additional 4 hrs. After labeling, the cells were washed twice with 5 mM MOPS, (3-[N-morpholino]propane sulfonic acid) solution containing 150 mM NaCl and 1 mM EDTA and collected in the lysis buffer containing 10% KOH and 80% (vol.) ethanol.

Cholesterol Extraction and Data Analysis:

In order to separate labeled cholesterol from labeled non-cholesterol lipids, the cells lysates were subject to saponification at 60° C. for 2 hrs. The lysates were then combined with 0.5 volume of H₂O and 2 volumes of hexane, followed by 30 minutes of vigorous shaking. After the separation of two phases, the upper-phase solution was collected and combined with 5 volumes of scintillation cocktail. The amount of ¹⁴C cholesterol was quantified by liquid scintillation counting. The IC₅₀ values were calculated with GraphPad software (Prism 3.03).

Compounds of the invention exhibit a range of IC₅₀ values of less than about 50 nM in the aforementioned cell assay. See, for example, the compound of: Example 2, which has an IC₅₀ of 0.27 nM.

C.) Protocol for Sterol Biosynthesis in L6 Rat Myoblast:

Cell Culture, Compounds Treatment and Cell Labeling:

L6 rat myoblast purchased from ATCC(CRL-1458) were grown in T-150 vented culture flasks and seeded on 12-well culture plates at a density of 60,000 cells per well. The cells were grown in DMEM, (Dulbecco's Modified Eagle Medium) (Gibco, #10567-014) containing 10% heat inactivated FBS (Fetal Bovine Serum) (Gibco # 10082-139) for 72 hours until reaching confluence. The cells were pre-incubated in media with compound and 0.2% DMSO (dimethyl sulfoxide) for 3 hours and then labeled by incubating in medium containing compound, 0.2% DMSO and 1 μCi/per mL of ¹⁴C acetic acid for an additional 3 hours. After labeling, the cells were washed once with 1×PBS (Gibco #14190-144) then lysed overnight at 4° C. in buffer containing 10% KOH and 78% (vol.) ethanol.

Cholesterol Extraction and Data Analysis:

Lipid ester bonds were hydrolyzed by saponification of the lysates at 60° C. for 2 hours. Sterols (including cholesterol) were extracted from saponified lysates by combining with 3 volumes of hexane and mixing by pipette 6 times. The upper organic phase solution was collected and combined with an equal volume of 1N KOH in 50% methanol and mixed by pipette 6 times. The upper organic phase was collected in a scintilant-coated plate (Wallac #1450-501) and hexanes removed by evaporation at room temperature for 3 hours. The amount of ¹⁴C cholesterol was quantified by scintillation counting in a Trilux 1450 plate reader (Wallac). The IC₅₀ values were calculated from % inhibitions relative to negative controls vs. compound concentration on Microsoft excel 2000 data analysis wizard using a sigmoid inhibition curve model with formula:
y=Bmax(1−(xⁿ/Kⁿ+xⁿ))+y2
Where K is the IC₅₀ for the inhibition curve, X is inhibitor concentration, Y is the response being inhibited and Bmax+Y2 is the limiting response as X approaches zero. Compounds of the invention have a L6 IC₅₀ value greater than about 100 nM in the aforementioned L6 Rat Myoblast. See, for example, the compound of: Example 1, which has an IC₅₀ of 449 nM

What is claimed is:

1. A compound having a Formula I, 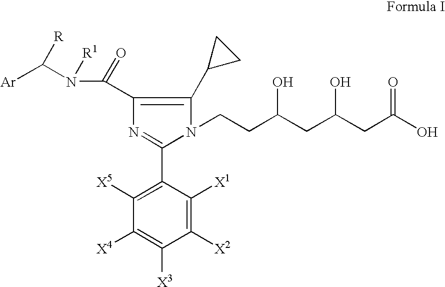
or a pharmaceutically acceptable salt, ester, amide, or stereoisomer thereof, wherein
Ar is an unsubstituted or substituted phenyl;
R is H or C₁-C₄ alkyl;
R¹ is H or methyl;
and X¹, X², X³, X⁴, and X⁵ are each independently selected from the group consisting of H, F, and Cl.

2. A compound according to claim 1 wherein X¹, X², X⁴, and X⁵ are H; and X³ is F.

3. A compound according to claim 1 wherein R is methyl.

4. A compound selected from the group consisting of
(3R,5R)-7-[5-Cyclopropyl-2-(4-fluoro-phenyl)-4-((R)-1-phenyl-ethylcarbamoyl)-imidazol-1-yl]-3,5-dihydroxy-heptanoic acid;
(3R,5R)-7-[5-Cyclopropyl-2-(4-fluoro-phenyl)-4-((S)-1-phenyl-ethylcarbamoyl)-imidazol-1-yl]-3,5-dihydroxy-heptanoic acid;
(3R,5R)-7-(5-Cyclopropyl-2-(4-fluoro-phenyl)-4-[methyl-((R)-1-phenyl-ethyl)-carbamoyl]-imidazol-1-yl)-3,5-dihydroxy-heptanoic acid;
7-[5-Cyclpropyl-2-(4-fluoro-phenyl)-4-(4-methyl-benzylcarbamoyl)-imidazol-1-y]-3,5-dihydroxy-heptanoic acid;
7-[5-Cyclopropyl-2-(4-fluoro-phenyl)-4-(4-methoxy-benzylcabamoyl)-imidazol-1-yl]-3,5-dihydroxy-heptanoic acid;
and pharmaceutically acceptable salts and lactone forms thereof.

5. A compound according to claim 1 wherein Ar is substituted with one or more groups selected from (C₁-C₆)alkyl, (C₁-C₆)alkoxy, F and Cl.

6. A stereoisomer of a compound of claim 1 comprising a (3R,5R)-isomer or the pharmaceutically acceptable salt, ester, or amide thereof.

7. A stereoisomer of a compound of claim 1 comprising a (3S,5R)-isomer or the pharmaceutically acceptable salt, ester, or amide thereof.

8. A stereoisomer of a compound of claim 1 comprising a (3R,5S)-isomer or the pharmaceutically acceptable salt, ester, or amide thereof.

9. A stereoisomer of a compound of claim 1 comprising a (3S,5S)-isomer or the pharmaceutically acceptable salt, ester or amide thereof.

10. A compound having a Formula II, 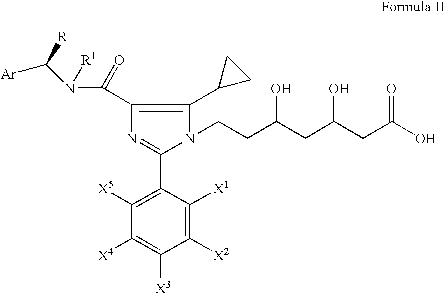
or a pharmaceutically acceptable salt, ester, amide, or stereoisomer thereof, wherein
Ar is an unsubstituted or substituted phenyl;
R is C₁-C₄ alkyl;
R¹ is H or methyl; and
X¹, X², X³, X⁴, and X⁵ are each independently selected from the group consisting of H, F and Cl.

11. A compound having a Formula III, 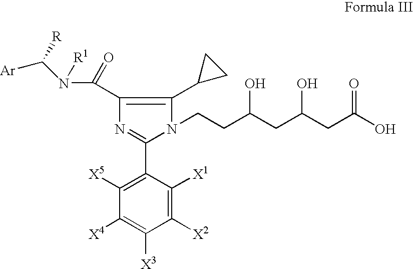
or a pharmaceutically acceptable salt, ester, amide, or stereoisomer thereof, wherein
Ar is an unsubstituted or substituted phenyl;
R is C₁-C₄ alkyl;
R¹ is H or methyl; and
X¹, X², X³, X⁴, and X⁵ are each independently selected from the group consisting of H, F and Cl.

12. A pharmaceutical composition comprising a compound of claim 1, the pharmaceutically acceptable salt, ester, amide, or stereoisomer or mixtures thereof; and a pharmaceutically acceptable carrier, diluent, or vehicle.
